(19) United States
(12) Plant Patent Application Publication
(10) Pub. No.: US 2002/0194656 P1
(43) Pub. Date: Dec. 19, 2002

(54) Ficus plant named 'Mini Amstel'

(75) Inventor: Sally Stribling, Homestead, FL (US)

Correspondence Address: Foley & Lardner, 3000 K Street, N.W., Suite 500, Washington, DC, 20007-5109, US

(73) Assignee: Miami Agra-Starts, Inc.

(21) Appl. No.: 09/880,880

(22) Filed: Jun. 15, 2001

(51) Int. Cl. A01H 5/00

(52) U.S. Cl. PLT/211

(57) ABSTRACT

A new and distinct cultivar of Ficus plant named ‘Mini Amstel’, as described and illustrated, and particulary characterized by the combined features of 40-45 mm wide leaves with elongated, lanceolate shape; medium green leaves closest to RHS 137 A; and irregular internodes with zig-zag pattern.

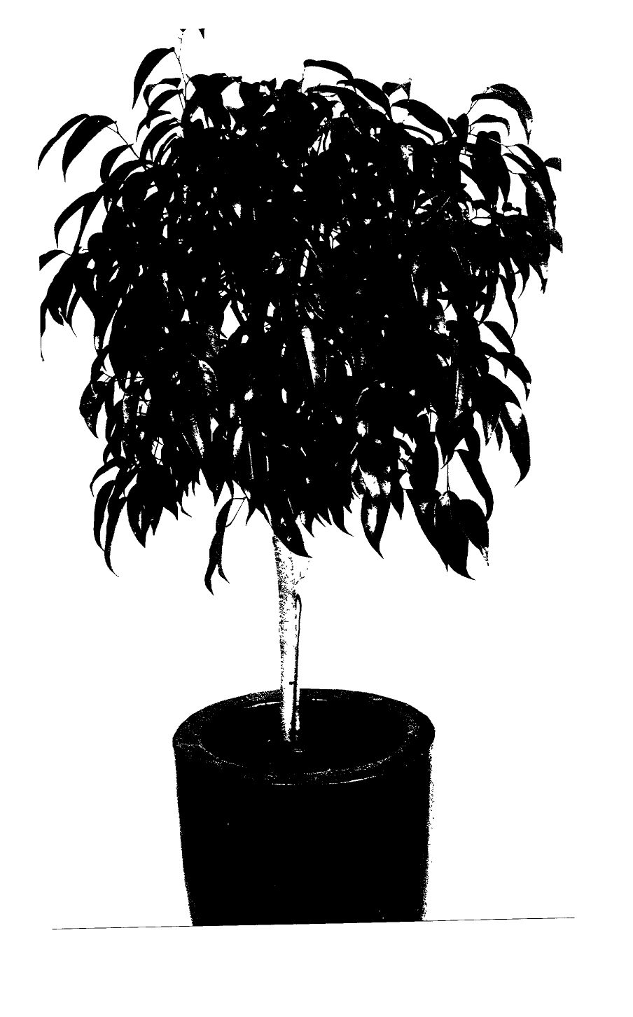

### BACKGROUND OF THE INVENTION

The present invention relates to a new and distinct cultivar of Ficus benjamina. The varietal denomination of the new cultivar is ‘Mini Amstel’. The new variety was discovered as a mutation in a controlled planting of Ficus benjamina in Homestead, Fla. in July in 1999.

The new variety is a mutation of the variety Ficus benjamina. The new variety was selected by Sally Stribling at a controlled outdoor planting in July, 1999. The new variety was first asexually propagated in October, 1999, in Homestead, Fla. Asexual reproduction through succeeding generations has established that the combination of characteristics as herein disclosed for the new cultivar are firmly fixed and retained through successive generations of asexual reproduction.

### BRIEF DESCRIPTION OF THE INVENTION

The following traits have been repeatedly observed and are determined to be basic characteristics of ‘Mini Amstel’ which in combination distinguish this Ficus as a new and distinct cultivar:

1. 40-45 mm wide leaves with elongated, lanceolate shape;

2. medium green leaves closest to RHS 137 A; and

3. irregular internodes with zig-zag pattern.

‘Mini Amstel’ varies from that of the parent plant in that the leaves are wider and have an elongated lanceolate shape. This gives the leaf a banana shape that is approximately half of the length of the Ficus variety ‘Amstel King’. The internodes are irregular in length with a slight zig-zag pattern. Please note that the Ficus ‘Amstel King’ and ‘Mini Amstel’ are not related as ‘Amstel King’ is a Macllelandii type Ficus and ‘Mini Amstel’ is a Ficus benjamina.

### BRIEF DESCRIPTION OF THE DRAWINGS

The accompanying photographic illustration is a photograph of a Ficus ‘Mini Amstel’ showing the characteristics of the foliage of a typical Ficus ‘Mini Amstel’ plant.

### DETAILED DESCRIPTION OF THE NEW VARIETY

‘Mini Amstel’ has not been observed under all possible environmental conditions. The phenotypic expression may vary with variations in environment such as temperature, light intensity, day length and cultural conditions, without a change in the genotype of the plant.

The following observations and descriptions are of plant grown in Homestead, Fla. in a shade house from October, 2000 until April, 2001. In this description, color references are to The Royal Horticultural Society Colour Chart (RHS). The color references are approximate as color depends to a degree on horticultural practices such as light level and fertilization rates.

### CLASSIFICATION

Botanical: Ficus benjamina ‘Mini Amstel’

Commercial: Ficus cv. Mini Amstel

PARENTAGE.—Ficus benjamina .

PROPAGATION.—By vegetative cuttings and other known asexual reproduction techniques.

### PLANT

Form.—An Upright woody multiple irregular branching Ficus with slightly zig-zagging internodes and elongated lanceolate shaped leaves

Growth.—Upright multiple branching

Height.—Height attained as of April, 2001, 1.5 meters. This height has been maintained by pruning in controlled plantings in a shade house in Homestead, Fla.

### FOLIAGE

Size.—7-11 cm long; 40-45 mm wide

Quantity.—Numerous multiple

Color.—Medium Green closest to RHS 137 A

Shape.—Lanceolate with Acuminate leaf tip

Texture.—Smooth and glabrous

Veination.—Single mid-rib vein under side of leaf from petiole to leaf tip typical of benjamina type Ficus

Edge.—Smooth

Serration.—None

Stipules.—None

Auricle.—None Petiole: Irregular between 15-35 mm.

DISEASE/INSECT RESISTANCE: No particular claim is made in reference to disease resistance. It has been observed to be susceptible to Thrips and Spider mites just as the parent plant Rachis; not apparent.

### WOOD

New Shoots.—Medium greyed-green closest to RHS 197B

Old Wood.—Greyed-green between 197C and 197D

INTERNODE SPACING: Irregular, between 15-35 mm

PREFERRED GROWING CONDITIONS: 25-35 degrees centigrade under 5,000 foot candles

REPRODUCTIVE ORGANS: None observed

I/We claim:

1. A new and distinct variety of ficus designated ‘Mini amstel’, substantially as illustrated and described:
